(19) United States
(12) Patent Application Publication
(10) Pub. No.: US 2005/0192443 A1
(43) Pub. Date: Sep. 1, 2005

(54) Biological response modifier for the treatment of cancer

(75) Inventor: Aiping H. Young, North York (CA)

Correspondence Address: HOUSTON ELISEEVA, 4 MILITIA DRIVE, SUITE 4, LEXINGTON, MA, 02421, US

(73) Assignee: Lorus Therapeutics Inc., Markham (CA)

(21) Appl. No.: 10/821,649

(22) Filed: Apr. 8, 2004

Related U.S. Application Data
(63) Continuation-in-part of application No. 10/416,259, filed on Jan. 2, 2004.

(30) Foreign Application Priority Data
Nov. 8, 2000 (CA) 2,325,361

(51) Int. Cl. A61K 31/4025; C07D 43/14

(52) U.S. Cl. 548/517; 514/422

(57) ABSTRACT

The present invention provides anticancer biological response modifier for the treatment of cancer. In accordance with an aspect of the present invention, there is provided a composition for the treatment of breast and prostate cancer in a mammal, said composition comprising small molecular weight components of less than 3000 daltons, and having the following properties: is extracted from bile of animals; is capable of stimulating monocytes and/or macrophages in vitro and/or in vivo; is capable of modulating tumor necrosis factor production and/or release; contains no measurable level of IL-1α, IL-1β, TNF, IL-6, IL-8, IL-4, GM-CSF or IFN-gamma; is not cytotoxic to human peripheral blood mononuclear cells; is not an endotoxin; and optionally with (ii) one or more anticancer agent(s), wherein said combination has therapeutic synergy or improves the therapeutic index in the treatment of cancer over the composition or the anticancer agent(s) alone. Another aspect of the present invention provides the use of this composition or combination in the manufacture of a medicament for the treatment of breast or prostate cancer in a mammal.

### CROSS REFERENCE TO RELATED APPLICATIONS

This application is a continuation-in-part of U.S. patent application Ser. No. 10/416,259 filed May 8, 2003, which, in turn, is a national stage application of PCT/CA01/01558 designating the U.S. and filed Nov. 8, 2001, which, in turn, claims priority to a Canadian patent application number CA2,325,361 filed Nov. 8, 2000, all of which are incorporated by reference herein in their entirety.

### FIELD OF THE INVENTION

The present invention relates to anticancer biological response modifiers alone or in combination with anticancer agents, pharmaceutical compositions comprising the same, and the use thereof in the treatment of cancer.

### BACKGROUND OF THE INVENTION

There are a number of therapies directed towards the treatment of cancer, including chemotherapeutic drugs, radiation, gene therapy and antisense oligonucleotides. One drawback to current therapies is the toxicity associated with most treatments. Moreover, oftentimes large dosages must be administered over an extended period of time in order to attain therapeutic benefit. Thus, a need remains for more effective treatments.

A bile extract has been prepared that is known to be able to modify the biological response of cells of the immune system. The production and characterization of this bile-derived Biological Response Modifier (BD-BRM) has been described in International Patent Application Serial No. PCT/CA94/00494, published Feb. 16, 1995 as WO 95/07089, International Patent Application Serial No. PCT/CA96/00152, published Sep. 19, 1996 as WO 96/28175 and U.S. Pat. No. 6,280,774. The use of this immunomodulatory composition as an anti-viral has been described in International Patent Application Serial No. PCT/CA98/00494, published Nov. 26, 1998 as WO 98/52585. These applications are herein incorporated by reference in their entirety.

The BD-BRM composition is composed of small molecular weight components of less than 3000 daltons, and has one or more of the following properties:
a) is extracted from bile of animals;
b) is capable of stimulating monocytes and/or macrophages in vitro and/or in vivo;
c) is capable of modulating tumor necrosis factor production and/or release;
d) contains no measurable level of IL-1α, IL-1β, TNF, IL-6, IL-8, IL-4, GM-CSF or IFN-γ;
e) shows no cytotoxicity to human peripheral blood mononuclear cells or lymphocytes; and
f) is not an endotoxin.

The bile-derived biologic response modifier (BD-BRM) is a composition that has been hypothesized to exert anti-tumour activity via the activation of macrophages, with subsequent enhancement of cell-mediated immune response to tumours. Its precise mechanism of action remains unknown.

The cumulative results of studies with BD-BRM revealed following:
(1) BD-BRM does not directly stimulate lymphocytes to synthesize DNA or undergo blastogenesis and cell division. BD-BRM does not directly stimulate the development of lymphocyte-mediated cytotoxicity.
(2) BD-BRM can stimulate normal peripheral blood monocytes to express cytocidal activity in a dose-dependent manner. The activity elicited by BD-BRM is equal to or greater than the activity produced in response to more conventional macrophage activators that are currently under investigation in cancer patients including: Gamma Interferon; Granulocyte-Monocyte Colony Stimulating Factor; Monocyte Colony Stimulating Factor; and Interleukin-12.
(3) BD-BRM can stimulate both the peripheral blood monocytes and regional, tumour-associated macrophages from cancer patients to express significant cytocidal activity. This included peritoneal macrophages from women with gynaecological malignancies and alveolar macrophages from patients with lung cancer. BD-BRM has been found to stimulate macrophages from cancer patients to kill autologous and heterologous tumour cells obtained from surgical specimens of patients. Of potentially greater importance is the finding that BD-BRM can often stimulate cancer patient macrophages that are unresponsive to stimulation with conventional activators such as gamma interferon+endotoxin.
(4) The hypersecretion of prostaglandins, both by macrophages and by tumor cells from cancer patients has been shown to be a principal cause of the immunosuppression seen in patients with advanced malignant disease. One determinant of the biological activity of different macrophage activators in cancer patients PBMs, therefore, is the sensitivity of the activator to arachidonic acid metabolism and the secretion by the cell of prostaglandins. The development of macrophage cytocidal function in response to BD-BRM was found to be insensitive to the inhibitory effects of prostaglandins. This is considered important therapeutically because the effectiveness of many other biological activators is limited by prostaglandins.
(5) BD-BRM can stimulate cytocidal function in macrophages obtained from cancer patients (including pancreatic cancer) who are undergoing cytotoxic therapy. Of note is the fact that BD-BRM was more effective in stimulating tumouricidal function than conventional activators such as gamma interferon plus endotoxin.
(6) BD-BRM can also stimulate cytocidal function in macrophages obtained from patients with Kaposi's sarcoma even at very late stages of the disease. Thus, the action of BD-BRM appears to be independent of the need for collaboration with other immune cell types including helper T-lymphocytes.
(7) The macrophage cytocidal function that develops in response to BD-BRM may be associated with the expression of TNFα by the macrophages. However, other mechanisms for cytotoxicity may also be involved. The BD-BRM composition from bovine sources promotes the release of TNF from human peripheral blood mononuclear cells and from the pre-monocyte cell line U-937 in what appears to be physiological quantities. Because TNF is known to initiate a cascade of inflammatory and antitumor cytokine effects, the composition could exert its antineoplastic effect by stimulating human leukocytes to release TNF (and possibly other cytokines).
(8) Demonstrates anti-tumour activity in a mouse tumour (plasmacytoma) model.
(9) Exhibits no toxicity in animals at doses up to 125× the doses used in human toxicity studies with no LD⁵⁰ yet reached in toxicity studies.
(10) Induces the phenomenon of apoptosis in some continuous cell lines.
(11) Is non-cytotoxic to human PBMNs and lymphocytes. The survival of human peripheral blood mononuclear cells (PBMNs) and lymphocytes is not affected by BD-BRM.

The central hypothesis guiding investigations of the BD-BRM composition is that the therapeutic efficacy of a powerful biological stimulator can depend on its ability to elicit suitable modulation of the immune system, such as by activating macrophages and/or monocytes to produce certain cytokines or promote activity to seek and remove or destroy disease-causing viruses or cells negatively affected by such viral infections. Such function could be generated by direct stimulation of resident immune cells in tumour microenvironments. Alternatively, this function could be generated by stimulation of circulating immune cells if those cells were then able to home in on tumour sites and to function in that environment.

This background information is provided for the purpose of making known information believed by the applicant to be of possible relevance to the present invention. No admission is necessarily intended, nor should be construed, that any of the preceding information constitutes prior art against the present invention. Publications referred to throughout the specification are hereby incorporated by reference in their entireties in this application.

### SUMMARY OF THE INVENTION

An object of the present invention is to provide anticancer biological response modifier therapies. In accordance with an aspect of the present invention, there is provided a use of a composition to treat breast or prostate cancer in a mammal comprising small molecular weight components of less than 3000 daltons, and having the following properties: is extracted from bile of animals; is capable of stimulating monocytes and/or macrophages in vitro and/or in vivo; is capable of modulating tumor necrosis factor production and/or release; contains no measurable level of IL-1α, IL-1β, TNF, IL-6, IL-8, IL-4, GM-CSF or IFN-gamma; is not cytotoxic to human peripheral blood mononuclear cells; and is not an endotoxin. Another aspect of the present invention provides the use of the composition in the manufacture of a medicament or a pharmaceutical kit for the treatment of breast or prostate cancer in a mammal.

In accordance with another aspect of the invention, there is provided a pharmaceutical kit for the treatment of breast or prostate cancer in a mammal comprising: (i) a dosage unit of a composition and a pharmaceutically acceptable carrier wherein the composition comprises small molecular weight components of less than 3000 daltons, and has the following properties: is extracted from bile of animals; is capable of stimulating monocytes and/or macrophages in vitro and/or in vivo; is capable of modulating tumor necrosis factor production and/or release; contains no measurable level of IL-1α, IL-1β, TNF, IL-6, IL-8, IL-4, GM-CSF or IFN-gamma; is not cytotoxic to human peripheral blood mononuclear cells; is not an endotoxin; and (ii) a dosage unit of one or more chemotherapeutic drug(s) and a pharmaceutically acceptable carrier, (i) and (ii) being provided in amounts that are effective, in combination, for killing tumour or metastatic cells.

In accordance with another aspect of the invention, there is provided a pharmaceutical composition for the treatment of breast or prostate cancer in a mammal comprising: (i) a composition comprising small molecular weight components of less than 3000 daltons, and having the following properties: is extracted from bile of animals; is capable of stimulating monocytes and/or macrophages in vitro and/or in vivo; is capable of modulating tumor necrosis factor production and/or release; contains no measurable level of IL-1α, IL-1β, TNF, IL-6, IL-8, IL-4, GM-CSF or IFN-gamma; is not cytotoxic to human peripheral blood mononuclear cells; is not an endotoxin; (ii) one or more chemotherapeutic drug(s); and (iii) a pharmaceutically acceptable carrier; wherein said pharmaceutical composition has therapeutic synergy or improves the therapeutic index in the treatment of cancer over the composition or the chemotherapeutic drug(s) alone.

In accordance with another aspect of the invention, there is provided a combination for use in the treatment of breast cancer or prostate cancer in a mammal, comprising: (i) a composition comprising small molecular weight components of less than 3000 daltons, and having the following properties: is extracted from bile of animals; is capable of stimulating monocytes and/or macrophages in vitro and/or in vivo; is capable of modulating tumor necrosis factor production and/or release; contains no measurable level of IL-1α, IL-1β, TNF, IL-6, IL-8, IL-4, GM-CSF or IFN-gamma; is not cytotoxic to human peripheral blood mononuclear cells; is not an endotoxin; and (ii) one or more anticancer agent(s), wherein said combination has therapeutic synergy or improves the therapeutic index in the treatment of cancer over the composition or the anticancer agent(s) alone.

In accordance with another aspect of the invention, there is provided a method for treating breast or prostate cancer in a mammal, comprising the step of administering a therapeutically effective amount of a composition comprising small molecular weight components of less than 3000 daltons, and having the following properties: is extracted from bile of animals; is capable of stimulating monocytes and/or macrophages in vitro and/or in vivo; is capable of modulating tumor necrosis factor production and/or release; contains no measurable level of IL-1α, IL-1β, TNF, IL-6, IL-8, IL-4, GM-CSF or IFN-gamma; is not cytotoxic to human peripheral blood mononuclear cells; is not an endotoxin. Another aspect of the present invention provides a method of treating breast or prostate cancer in a mammal in combination with one or more anticancer agent(s), wherein said combination has therapeutic synergy or improves the therapeutic index in the treatment of breast or prostate cancer over the composition or the anticancer agent(s) alone.

### BRIEF DESCRIPTION OF THE DRAWINGS

Further details of the invention are described below with the help of the examples illustrated in the accompanying drawings in which:

FIG. 1 is a graph showing dose response of the composition of the invention in stimulating peripheral blood monocyte function.

FIG. 2 illustrates the growth of Human Pancreatic Adenocarcinoma (BxPC-3) in CD-1 Nude Mice.

FIG. 3 illustrates the weight of Human Pancreatic Adenocarcinoma (BxPC-3) in CD-1 Nude Mice.

FIG. 4 illustrates the growth of Human Pancreatic Carcinoma (SU.86.86.) in CD-1 Nude Mice.

FIG. 5 illustrates the weight of Human Pancreatic Carcinoma (SU.86.86.) in CD-1 Nude Mice.

FIG. 6 illustrates the growth of Human Melanoma(A2058) in CD-1 Nude Mice.

FIG. 7 illustrates the weight of Human Melanoma(A2058) in CD-1 Nude Mice.

FIG. 8 illustrates the growth of Human Melanoma(C8161) in CD-1 Nude Mice.

FIG. 9 illustrates the weight of Human Melanoma(C8161) in CD-1 Nude Mice.

FIG. 10 illustrates the growth of Human Breast Adenocarcinoma(MDA-MB-231) in CD-1 Nude Mice.

FIG. 11 illustrates the weight of Human Breast Adenocarcinoma (MDA-MB-231) in CD-1 Nude Mice.

FIG. 12 illustrates the growth of Human Breast Adenocarcinoma (MDA-MB-231) in CD-1 Nude Mice.

FIG. 13 illustrates the weight of Human Breast Adenocarcinoma (MDA-MB-231) in CD-1 Nude Mice.

FIG. 14 illustrates the growth of Human Prostate Carcinoma (PC-3) in SCID Mice.

FIG. 15 illustrates the weight of Human Prostate Carcinoma (PC-3) in SCID Mice.

FIG. 16 illustrates the growth of Human Pancreatic Carcinoma (BxPC-3) in CD-1 Nude Mice.

FIG. 17 illustrates the weight of Human Pancreatic Carcinoma (SU.86.86) in CD-1 Nude Mice.

FIG. 18 illustrates the growth of Human Prostate Carcinoma (DU145) in SCID Mice.

FIG. 19 illustrates the weight of Human Prostate Carcinoma (DU145) in SCID Mice.

FIG. 20 illustrates the growth of Human Ovary Adenocarcinoma (SK-OV-3) in CD-1 Nude Mice.

FIG. 21 illustrates the growth of Human Ovary Adenocarcinoma (SK-OV-3) in CD-1 Nude Mice.

FIG. 22 illustrates the growth of Human Lung Adenocarcinoma (H460) in CD-1 Nude Mice.

FIG. 23 illustrates the weight of Human Lung Adenocarcinoma (H460) in CD-1 Nude Mice.

FIG. 24 illustrates the growth of Human Small Cell Lung Carcinoma (H209) in SCID Mice.

FIG. 25 illustrates the weight of Human Small Cell Lung Carcinoma (H209) in SCID Mice.

FIG. 26 illustrates the anticancer activities of BD-BRM and clinical use drugs in a panel of human breast, ovarian and prostate xenografted tumors.

FIG. 27 illustrates the stimulation of cytotoxicity of peritoneal macrophages by BD-BRM against various human breast, ovarian and prostate cancer cell lines.

### DETAILED DESCRIPTION OF THE INVENTION

The present invention provides anticancer biological response modifier (BD-BRM) for the treatment of breast or prostate cancer in a mammal. The BD-BRM composition comprises small molecular weight components of less than 3000 daltons, and has the following properties: it is extracted from bile of animals; it is capable of stimulating monocytes and/or macrophages in vitro and/or in vivo; it is capable of modulating tumor necrosis factor production and/or release; it contains no measurable level of IL-1α, IL-1β, TNF, IL-6, L-8, IL-4, GM-CSF or IFN-gamma; it is not cytotoxic to human peripheral blood mononuclear cells; it is not an endotoxin; and can optionally be used in combination with one or more anticancer agent(s), wherein the BD-BRM combination has therapeutic synergy or improves the therapeutic index in the treatment of cancer over the composition or the anticancer agent(s) alone. The present invention further provides the use for the manufacture of a medicament or a pharmaceutical kit and in the treatment of breast or prostate cancer in a mammal.

Components of the BD-BRM and Combinations

BD-BRM Composition

Experimental evidence to date indicates that the unique immunomodulatory properties of BD-BRM activity are associated with low molecular weight material derived from bile. The BD-BRM composition of the present invention comprises small molecular weight components of less than 3000 daltons, and having at least one of the following properties:
a) is extracted from the bile of animals;
b) is capable of stimulating or activating monocytes and/or macrophages in vitro and/or in vivo;
c) is capable of modulating tumor necrosis factor production and/or release;
d) contains no measurable level of IL-1α, IL-1β, TNF, IL-6, IL-8, IL4, GN-CSF or IFN-γ;
e) shows no cytotoxicity to human peripheral blood mononuclear cells or lymphocytes; and
f) is not an endotoxin.

As mentioned above, the production and characterization of the BD-BRM composition has been described in preceding patent applications, and is also summarized in Example 1. The composition can be produced in a consistently reproducible form using the method as generally described above with demonstrated identity, potency and purity from batch to batch. Identity and purity are determined using reverse-phase high pressure liquid chromatography. (See Example 1). The compositions have a consistently reproducible pattern on reverse-phase HPLC. The composition may be used in a concentrated form. The composition may also be lyophilized. The composition may be used without further modification by simply packaging it in vials and sterilizing.

The compositions are also characterized by the properties hereinbefore mentioned, for example their ability to stimulate monocytes and macrophages in vitro and in vivo, etc. The compositions activate PBMNs to release TNF in vitro as measured by the Monocyte/Macrophage Activation Assay (TNF-Release) as described in Example 2.

Anticancer Agents

This invention provides for a BD-BRM composition that can optionally be used in combination with one or more other anticancer agents. An Aanticancer agent@ is any compound, composition or treatment that prevents or delays the growth and/or metastasis of cancer cells. Such anticancer agents include but are not limited to chemotherapeutic drug treatment, radiation, gene therapy, hormonal manipulation, immunotherapy and antisense oligonucleotide therapy. It is to be understood that anticancer agents for use in the this invention also include novel compounds or treatments developed in the future that can be used to generate therapeutic combinations as described herein.

Examples of candidate anti-cancer compounds that may be useful in the combinations of this invention are: antisense sequences, Drugs for Promyelocytic Leukemia: Tretinoin (Vesanoid7); Drugs for Chronic Myeloid Leukemia: Low-dose Interferon (IFN)-alpha; Drugs Used in Gastric Cancer: Antibiotics, Antineoplastics; Acute Lymphoblastic Leukemia: Pegaspargase (Oncaspar7), Rhone-poulenc Rorer, L-asparaginase, Il-2; Drugs for Colon Cancer: Edatrexate or 10-ethyl-10-deaza-aminopterin or 10-edam, 5-fluorouracil (5-FU) and Levamisole, Methyl-ccnu (Methyl-chloroethyl-cyclohexyl-nitrosourea), Fluorodeoxyuridine (Fudr), Vincristine; Drugs for Esophageal Cancer: Porfimer Sodium (Photofrin7), Quadra Logic Technologies, or Treatment with a Neodymium:yag (Nd:yag7) Laser; Drugs Used in Colorectal Cancer: Irinotecan (Camptosar7), Pharmacia & Upjohn, Topotecan (Hycamtin7), Loperamide (Imodium7), 5-fluorouracil (5-FU); Drugs For Advanced Head and Neck Cancers: Docetaxel (Taxotere7); Drugs for Non-hodgkin's Lymphoma: Rituximab, Etoposide; Drugs for Non-small-cell Lung Cancer: A Vinca Alkaloid, Vinorelbine Tartrate (Navelbine7), Wellcome, Paitaxel, (Taxol7), Docetaxel (Taxotere7), Topotecan, Irinotecan, Gemcitabine; Drugs for Ovarian Cancer: Docetaxel (Taxotere7), Gemcitabine, (Gemzar7), Irinotecan (Camptosar7), Paclitaxel (Taxol7), Topotecan (Hycamtin7), Amifostine (Ethyol7), Us Bioscience (For Reducing the Cumulative Renal Toxicity Associated with Repeated Cisplatin Therapy in Patients with Advanced Ovarian Cancer); Drugs to Prevent Melanoma (Sun Screens): 2-ethylhexyl-p-methoxy-cinnamate (2-ehmc), Octyl-N-dimethyl-p-aminobenzoate (O-paba), Benzophenone-3 (Bp-3); Drugs for Prostate Cancer: Flutamide (Eulexin7), Finasteride (Proscar7), Terazosin (Hytrin7), Doxazosin (Cardura7), Goserelin Acetate (Zoladex7), Liarozole, Nilutamide (Nilandron7), Mitoxantrone (Novantrone7), Prednisone (Deltasone7); Drugs for Pancreatic Cancer: Gemcitabine (Gemzar7), 5-fluorouracil; Drugs for Advanced Renal Cancer: Interleukin-2 (Proleukin7), Chiron Corp.; Additional Anti-neoplastic Drugs: Porfimer Sodium, Axcan, Dacarbazine, Faulding, Etoposide, Faulding, Procarbazine HCl, Sigma-tau, Rituximab, Roche, Paclitaxel (Taxol7), Bristol-myers Squibb, Trastuzumab (Herceptin7), Roche, Temozolomide (Temodal7), Schering; Alkylating Agents Used in Combination Therapy for Different Cancers: Cyclophosphamide, Cisplatin, Melphalan.

Antisense Compounds

The specificity and sensitivity of antisense compounds makes them useful in diagnostics, therapeutics, prophylaxis, as research reagents and in kits. In the context of the present invention, the terms “antisense compound” and “antisense oligonucleotide” each refer to an oligomer or polymer of ribonucleic acid (RNA), or deoxyribonucleic acid (DNA), or mimetics thereof. These terms also include chimeric antisense compounds, which are antisense compounds that contain two or more chemically distinct regions, each made up of at least one monomer unit. In accordance with the present invention, the terms “antisense compound” and “antisense oligonucleotide” further include oligonucleotides composed of naturally occurring nucleobases, sugars and covalent internucleoside (backbone) linkages, as well as oligonucleotides comprising non-naturally-occurring moieties that function similarly. Such modified or substituted oligonucleotides are well known to workers skilled in the art and often preferred over native forms because of desirable properties such as, for example, enhanced cellular uptake, enhanced affinity for nucleic acid target and increased stability in the presence of nucleases. The antisense compounds in accordance with the present invention comprise from about 7 to about 50 nucleobases, or from about 7 to about 30. Alternatively, the antisense compounds comprise a mixture of short oligomers which will bind to the target nucleic acid in tandem (i.e. they are complementary to sequences that are adjacent to one another in the target nucleic acid).

Examples of antisense compounds useful in the present invention include oligonucleotides containing modified backbones or non-natural internucleoside linkages. In accordance with the present invention, oligonucleotides having modified backbones include those that retain a phosphorus atom in the backbone and those that do not have a phosphorus atom in the backbone. For the purposes of the present invention, and as sometimes referenced in the art, modified oligonucleotides that do not have a phosphorus atom in their internucleoside backbone can also be considered to be oligonucleosides.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may be additionally or alternatively employed. Similar techniques using phosphorothioates and alkylated derivatives have been employed to produce oligonucleotides.

Antisense oligonucleotides have been successfully employed as therapeutic moieties in the treatment of disease states such as cancer. Antisense compounds exert their effects by specifically modulating expression of a gene implicated in a specific disease state. Thus, the present invention contemplates the therapeutic administration of an effective amount of a combination of the BD-BRM composition of the present invention and an appropriate antisense compound to a mammal suspected of having a disease or disorder which can be treated by specifically modulating gene expression. The present invention further contemplates the prophylactic use of a combination of the BD-BRM composition and an antisense compound in the prevention of a cancer which is related to over- or under-expression of a specific gene.

Pharmaceutical Compositions

The combinations of the present invention may be converted using customary methods into pharmaceutical compositions. The pharmaceutical composition contains the BD-BRM or combinations of the invention, either alone or together with other active or inactive substances. Such pharmaceutical compositions can be for oral, topical, rectal, parenteral, local, inhalant, or intracerebral use. They are therefore in solid or semisolid form, for example pills, tablets, creams, gelatin capsules, capsules, suppositories, soft gelatin capsules, gels, membranes, and tubelets. For parenteral and intracerebral uses, those forms for intramuscular or subcutaneous administration can be used, or forms for infusion or intravenous or intracerebral injection can be used, and can therefore be prepared as solutions of the combinations or as powders of the combinations to be mixed with one or more pharmaceutically acceptable excipients or diluents, suitable for the aforesaid uses and with an osmolarity that is compatible with the physiological fluids. For local use, those preparations in the form of creams or ointments for topical use or in the form of sprays may be considered; for inhalant uses, preparations in the form of sprays, for example nose sprays, may be considered. Preferably, the BD-BRM composition or the combinations are administered intramuscularly.

The pharmaceutical compositions can be prepared by per se known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (Nack Publishing Company, Easton, Pa., USA 1985).

On this basis, the pharmaceutical compositions include, albeit not exclusively, the combination of the invention in association with one or more pharmaceutically acceptable vehicles or diluents, and are contained in buffered solutions with a suitable pH and iso-osmotic with the physiological fluids.

The compositions and agents of the invention are intended for administration to humans or animals.

The dosage requirements of the pharmaceutical compositions according to the present invention will vary with the particular BD-BRM or combinations employed, the route of administration and the particular cancer and cancer patient being treated. Treatment will generally be initiated with small dosages less than the optimum dose of the compound. Thereafter the dosage is increased until the optimum effect under the circumstances is reached. In general, the pharmaceutical compositions according to the present invention are most administered at a concentration that will generally afford effective results without causing any harmful or deleterious side effects. The compounds can be administered either as a single unit dose, or if desired, the dosage can be divided into convenient subunits administered at suitable times throughout the day. The amount of the pharmaceutical composition that will be effective in treatment can be determined by standard clinical techniques, known to a worker skilled in the art [for example, see Remington's Pharmaceutical Sciences, 18^{th} Edition, Mack Publishing Co., Easton, Pa. (1990)].

Therapeutic Activity of the Combinations

The combinations of the present invention have a net anticancer effect that is greater than the anticancer effect of the individual components of the combination when administered alone. The anticancer effect is increased without a concomitant increased toxic effect. Without being limited by mechanism, by combining one or more anticancer agents with a BD-BRM composition it is possible to:
(12) increase the therapeutic effect of the anticancer agent(s);
(13) increase the therapeutic effect of the BD-BRM composition;
(14) decrease or delay the toxicity phenomena associated with the anticancer agent(s); and/or
(15) decrease or delay the toxicity phenomena associated with the BD-BRM composition, in comparison to treatment with the individual components of the combination.

In one embodiment the combination of the present invention provides an improved efficacy, over treatment using the components of the combination alone, that may be demonstrated by determination of the therapeutic synergy.

A combination manifests therapeutic synergy if it is therapeutically superior to one or other of the constituents used at its optimum dose [T. H. Corbett et al., (1982) Cancer Treatment Reports, 66, 1187]. To demonstrate the efficacy of a combination, it may be necessary to compare the maximum tolerated dose of the combination with the maximum tolerated dose of each of the separate constituents in the study in question. This efficacy may be quantified using techniques and equations commonly known to workers skilled in the art. [T. H. Corbett et al., (1977) Cancer, 40, 2660.2680; F. M. Schabel et al., (1979) Cancer Drug Development, Part B, Methods in Cancer Research, 17, 3-51, New York, Academic Press Inc.].

The combination, used at its own maximum tolerated dose, in which each of the constituents will be present at a dose generally not exceeding its maximum tolerated dose, will manifest therapeutic synergy when the efficacy of the combination is greater than the efficacy of the best constituent when it is administered alone.

In another embodiment the combination of the present invention improves the therapeutic index in the treatment of cancer over that of the BD-BRM composition or the anticancer agent(s) when administered to a patient alone.

A median effective dose (ED₅₀) of a drug is the dose required to produce a specified effect in 50% of the population. Similarly, the median lethal dose (LD₅₀) of a drug, as determined in preclinical studies, is the dose that has a lethal effect on 50% of experimental animals. The ratio of the LD₅₀ to the ED₅₀ can be used as an indication of the therapeutic index. Alternatively the therapeutic index can be determined based on doses that produce a therapeutic effect and doses that produce a toxic effect (e.g. ED₉₀ and LD₁₀, respectively). During clinical studies, the dose, or the concentration (e.g. solution, blood, serum, plasma), of a drug required to produce toxic effects can be compared to the concentration required for the therapeutic effects in the population to evaluate the clinical therapeutic index. Methods of clinical studies to evaluate the clinical therapeutic index are well known to workers skilled in the art.

In one embodiment the combination of the present invention provides an improved therapeutic index, in comparison to that of the individual components of the combination when administered alone, by decreasing the observed LD₅₀ of at least one of the one or more anticancer agents in the combination.

In a related embodiment the combination of the present invention provides an improved therapeutic index, in comparison to that of the individual components of the combination when administered alone, by increasing the observed ED₅₀ of at least one of the one or more anticancer agents in the combination. In a further embodiment the combination of the present invention provides an improved therapeutic index, in comparison to that of the individual components of the combination when administered alone, by increasing the observed ED₅₀ of the bile-derived biological response modifier.

In another embodiment the efficacy of a combination according to the present invention may also be characterized by adding the actions of each constituent.

In order to prepare a combination according to the present invention one first selects one or more candidate anticancer agent(s) and measure its efficacy in a model of a cancer of interest, as would be well understood by one skilled in the art. The next step may be to perform a routine analysis to compare the efficacy of the one or more anticancer agent(s) alone to the efficacy of the one or more anticancer agent(s) in combination with varying amounts of the BD-BRM composition. Successful candidates for use in the combinations of the present invention will be those that demonstrate a therapeutic synergy with the BD-BRM or that improve the therapeutic index in comparison to the therapeutic index of the candidate agent(s).

The efficacy of the combinations of the present invention may be determined experimentally using standard techniques using cancer models well known to workers skilled in the art. Such cancer models allow the activity of combinations to be tested in vitro and in vivo in relation to the cancer of interest. Exemplary methods of testing activity are described in the Examples provided herein, although, it should be understood that these methods are not intended to limit the present invention.

One example of a method for studying the efficacy of the combinations on solid tumors in vivo involves the use of subject animals, generally mice, that are subcutaneously grafted bilaterally with 30 to 60 mg of a tumor fragment on day 0. The animals bearing tumors are mixed before being subjected to the various treatments and controls. In the case of treatment of advanced tumors, tumors are allowed to develop to the desired size, animals having insufficiently developed tumors being eliminated. The selected animals are distributed at random to undergo the treatments and controls. Animals not bearing tumors may also be subjected to the same treatments as the tumor-bearing animals in order to be able to dissociate the toxic effect from the specific effect on the tumor. Chemotherapy generally begins from 3 to 22 days after grafting, depending on the type of tumor, and the animals are observed every day. The different animal groups are weighed 3 or 4 times a week until the maximum weight loss is attained, and the groups are then weighed at least once a week until the end of the trial.

The tumors are measured 2 or 3 times a week until the tumor reaches approximately 2 g, or until the animal dies if this occurs before the tumor reaches 2 g. The animals are autopsied when sacrificed. The antitumour activity is determined in accordance with various recorded parameters.

In one example, breast cancer cell lines include but are not limited to Hs 566(B).T, Hs 574.T, Hs 574.SK, Hs 579.Mg, Hs 605.T, Hs 606, Hs 617.Mg, MB 157, B6.2, B25.2, B72.3, B38.1, CLN H11.4, As33, BT-483, Hs 578Bst, Hs 578T, MDA-MB-330, MDA-MB415, ZR-75-1, ZR-75-30, UACC-812, UAC-893, 786-O, CCD-1068Sk, HCC38, HCC70, HCC202, HCC1007 BL, HCC1008, HCC1143, HCC1187, HCC1395, HCC1395 BL, HCC 1419, HCC1428 BL, HCC1500, HCC1569, HCC1599, HCC1599 BL, HCC 1806, HCC1806, HCC1937, HCC1937 BL, HCC1954, HCC1964 BL, HCC2157, HCC2157 BL, HCC2218, HC38 BL, AU565, HCC1143 BL, HCC2218 BL, 4T1, CCD-1129SK, EMT6, Hs 402.Sk, Hs 540.T, MDA-MB-435S, MDA-MB-436, MDA-MB-453, MDA-MB-468, T-47D, Hs 294T, BT-20, BT-474, CAMA-1, MDA-MB-134-VI, MDA-MB-157, MDA-MB-175-VII, MDA-MB-231, MDA-MB-361 or ME-180.

In one example, the prostate cancer cell lines include but are not limited to PC-3, LNCaP; AT3B-1; MLLB-2; 22Rv1; NCI-H660; CLNH5.5; CLN H11.4; T-47D; DU 145

For a study of the combinations on leukaemias, the animals are grafted with a particular number of cells, and the antitumour activity is determined by the increase in the survival time of the treated mice relative to the controls.

Administration of the BD-BRM or Combinations

The uses and methods of the present invention comprise administering to a subject in need thereof an effective amount of a BD-BRM composition alone or in combination with one or more anticancer agents to a subject. As used herein, combination components are said to be administered in combination when the two or more components are administered simultaneously or are administered independently in a fashion such that the components will act at the same time.

Components administered independently can, for example, be administered separately (in time) or concurrently. Separately in time means at least minutes apart, and potentially hours, days or weeks apart. The period of time elapsing between the administration of the components of the combination of the invention can be determined by a worker of skill in the art, and will be dependent upon, for example, the age, health, and weight of the recipient, nature of the combination treatment, side effects associated with the administration of other component(s) of the combination, frequency of administration(s), and the nature of the effect desired. Components of the combinations of the invention may also be administered independently with respect to location and, where applicable, route of administration.

In one embodiment, an effective amount of a therapeutic composition comprising a BD-BRM composition, and a pharmaceutically acceptable carrier is administered to a subject. The BD-BRM composition or the pharmaceutical composition of the invention can be administered before during or after other anticancer treatment(s), or treatments for other diseases or conditions. For example a drug to treat adverse side effects of the anticancer treatment(s) can be administered concurrently with a combination of the invention or a pharmaceutical composition of the invention.

In another embodiment, an effective amount of a therapeutic composition comprising a BD-BRM composition and one or more anticancer agents, and a pharmaceutically acceptable carrier is administered to a subject. The combination or the pharmaceutical composition of the invention can be administered before during or after other anticancer treatment(s), or treatments for other diseases or conditions. For example a drug to treat adverse side effects of the anticancer treatment(s) can be administered concurrently with a combination of the invention or a pharmaceutical composition of the invention.

As indicated above the components of the combination of the present invention may be administered separately, concurrently, or simultaneously. In the case of separate administration the BD-BRM composition may be administered before, during or after administration of the anticancer agent(s). Furthermore, it would be readily apparent to a worker skilled in the art that the route of administration of each component of the combination is selected in order to maximize the therapeutic benefit of the component and it is not necessary that each component be delivered via the same route. The BD-BRM composition and/or the anticancer agent(s) of the combination may be administered via a single dose or via continuous perfusion.

The agents, compounds and compositions of this invention can be utilised in vivo, ordinarily in mammals, such as humans, sheep, horses, cattle, pigs, dogs, cats, rats and mice, or in vitro to treat cancer or cancer cells.

Cancers

As used herein, “cancer” refers to all types of cancer or neoplasm or malignant tumors found in mammals, including carcinomas and sarcomas. Examples of cancers are cancer of the brain, breast, cervix, colon, head and neck, kidney, lung, non-small cell lung, melanoma, mesothelioma, ovary, sarcoma, stomach, uterus, prostate and Medulloblastoma.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas include chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

In one embodiment of the invention, the BD-BRM of the invention alone or in combination with one or more anticancer agent(s) is used in the treatment a human breast cancer, wherein said breast cancer may be selected from the group of including but not limited to: intraductal carcinoma with or without Paget's disease; lobular carcimona in situ; invasive ductal carcinoma with or without Paget's disease; invasive lobular carcinoma; medullary carcinoma; colloid carcinoma (mucinous carcinoma); tubular carcinoma; adenoid cystic carcinoma; apocrine carcinoma or invasive papillary carcinoma.

In another embodiment of the invention, the BD-BRM of the invention alone or in combination with one or more anticancer agent(s) may be used in the treatment of human prostate cancer, wherein said prostate cancer may be selected from the group of prostate cancers including but not limited to: adenocarcinoma, a mucinous or signet ring tumor, adenoid cystic carcinoma, carcinoid, large prostatic duct carcinoma (including the endometrial type), or small cell undifferentiated cancer.

Pharmaceutical Kits

The present invention additionally provides for therapeutic kits containing (i) a dosage unit of a composition and a pharmaceutically acceptable carrier wherein the composition comprises small molecular weight components of less than 3000 daltons, and has the following properties: is extracted from bile of animals; is capable of stimulating monocytes and/or macrophages in vitro and/or in vivo; is capable of modulating tumor necrosis factor production and/or release; contains no measurable level of IL-1α, IL-1β, TNF, IL-6, IL-8, IL-4, GM-CSF or IFN-gamma; is not cytotoxic to human peripheral blood mononuclear cells; is not an endotoxin; and (ii) dosage unit of one or more chemotherapeutic drug(s) and a pharmaceutically acceptable carrier, said (i) and (ii) being provided in amounts that are effective, in combination, for selectively killing tumor or metastatic cells.

As used herein, a “dosage unit” is a pharmaceutical composition or formulation comprising at least one active ingredient and optionally one or more inactive ingredient(s). The dosage unit can be unitary, such as a single pill or liquid, containing all of the desired active ingredients and the inactive ingredients necessary and desired for making a dosage suitable for administration (e.g., tabletting compounds such as binders, fillers, and the like); the dosage unit can consist of a number of different dosage forms (e.g., pili(s) and/or liquid(s)) designed to be taken simultaneously as a dosage unit.

The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

### EXAMPLES

A worker skilled in the art can produce BD-BRM compositions, and assay BD-BRM compositions for activities such as in vitro and/or in vivo monocyte and/or macrophage stimulation, modulation of tumor necrosis factor production and/or release, content of IL-1α, IL-1β, TNF, IL-6, IL-8, IL-4, GM-CSF or IFN-gamma and endotoxin and cytotoxicity to human peripheral blood mononuclear cells, using the methods described in International Patent Application Serial No. PCT/CA94/00494, published Feb. 16, 1995 as WO 95/07089.

### Example 1

### In Vivo Evaluation of Efficacy of BD-BRM in the Treatment of Human Pancreatic Adenocarcinoma in Cd-1 Nude Mice

The mouse xenograft model of neoplasia was used in these studies to demonstrate the effect of treatment with a BD-BRM composition on tumor growth in mice. For comparison, separate groups of mice were treated with saline (control), a conventional chemotherapeutic drug or concurrently with a combination of a BD-BRM composition and a chemotherapeutic drug.

A human carcinoma cell line was grown as monolayer culture in Minimum essential medium (α-MEM) supplemented with 10% fetal bovine serum (FBS), 0.1 mM non-essential amino acid, 1.0 mM sodium pyruvate, 100 U/ml penicillin, 100 μg/ml streptomycin and 0.25 μg/ml amphotericin B and 2 mM L-alanyl-1-glutamine at 37° C. in an atmosphere of 5% CO₂ in air. The tumor cells were routinely subcultured twice weekly by trypsin-EDTA treatment. The cells were harvested from subconfluent logarithmically growing culture by treatment with trypsin-EDTA and counted for tumor inoculation. The cell lines used in the experiments herein are listed hereafter, though any carcinoma cell line capable of tumor formation upon inoculation could be used:
pancreatic adenocarcinoma (BxPC-3) (a gemcitabine-resistant cell line)
melanoma (A2058)
melanoma(C8161)
breast adenocarcinoma(MDA-MB-231)
prostate carcinoma (PC-3)
ovary adenocarcinoma (SK-OV-3)
large cell lung adenocarcinoma (H460)
small cell lung carcinoma (H209).

Tumor Inoculation: An acclimation period of at least 7 days was allowed between receipt of the immunocompromised animal and its inoculation. Typically CD-1 or SCID mice were used. When the female mice were 6-9 (most typically 6-7) weeks of age, each mouse was subcutaneously injected in the right flank with 3-10 million human carcinoma cells in 0.1 ml of PBS. Inoculated animals were divided into equal sized treatment groups of 9-20 (typically about 10) mice each and treated daily with saline (0.2 ml/mouse/day, i.p.), BD-BRM (0.2 ml/mouse/day, i.p.), a chemotherapeutic drug, or concurrently with BD-BRM (0.2 ml/mouse/day, i.p.) and a chemotherapeutic drug. The drug doses used in the experiments herein are listed hereafter, though any chemotherapeutic drug(s) or other anticancer agent(s) could be used:
gemcitabine (100 mg/kg in 0.1 ml saline/mouse/3 day, i.v.)
dacarbazine (DTIC) (80 mg/kg in 0.1 ml saline/mouse/day, i.p.)
taxol (10 mg/kg/week, i.v.)
5-fluorouracil
taxotere
cisplatin
mitoxanthrone (i.v.)

Tumour sizes were measured every other day in two dimensions using a caliper, and the volume was expressed in mm³ using the formula: V=0.5 a×b², where a and b are the long and short diameters of the tumor, respectively. Mean tumor volumes calculated from each measurement were then plotted in a standard graph to compare the anti-tumor efficacy of drug treatments to that of control. A day after the last treatment, tumors were excised from the animals and their weights were measured. The data are displayed as a tumour growth curve, and a bar graph showing mean tumor weights.

Mouse xenograft experiments with BD-BRM compositions
and BD-BRM combinations

| FIG. | Human | | Mouse | | combination | # mice with total |
|---|---|---|---|---|---|---|
| # | carcinoma | cell line | strain | drug | expt | tumor regression |
| 2, 3 | pancreatic | | CD-1 | gemcitabine | — | BRM: 4 (of 9) |
| 4, 5 | pancreatic | SU.86.86 | CD-1 | gemcitabine | gemcitabine | |
| 6, 7 | melanoma | A2058 | CD-1 | dacarbazine | dacarbazine | |
| 8, 9 | melanoma | C8161 | CD-1 | — | dacarbazine | comb: 5 (of 10) |
| 10, 11 | breast | MDA-MB-23 | CD-1 | Taxol | Taxol | |
| 12, 13 | breast | MDA-MB-23 | CD-1 | Taxol | Taxol | BRM: 2; comb: 5 (of 10) |
| 14, 15 | prostate | PC-3 | SCID | mitoxantrone | — | |
| 16 | pancreatic | BxPC-3 | CD-1 | 5-fluorouracil | 5-fluorouracil | comb: (5 of 10) |
| 17 | pancreatic | SU.86.86 | CD-1 | 5-fluorouracil | 5-fluorouracil | |
| 18, 19 | prostate | DU145 | SCID | mitoxantrone | — | |
| 20 | ovarian | SK-OV-3 | CD-1 | cisplatin | cisplatin | |
| 21 | ovarian | SK-OV-3 | CD-1 | taxol | taxol | |
| 22, 23 | lung, large | H460 | CD-1 | taxotere | taxotere | |
| | cell | | | | | |
| 24, 25 | lung, small | H209 | SCID | — | — | |

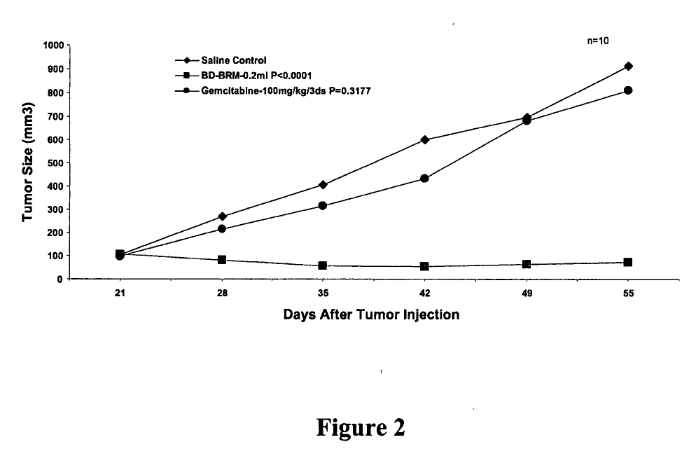

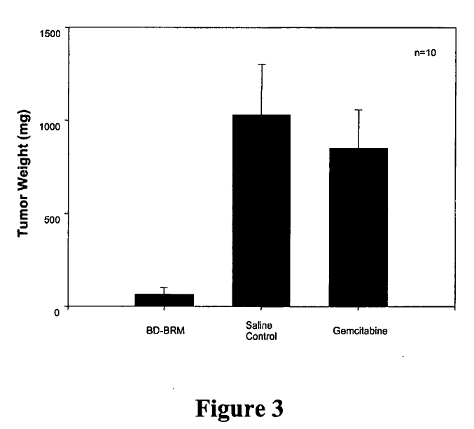

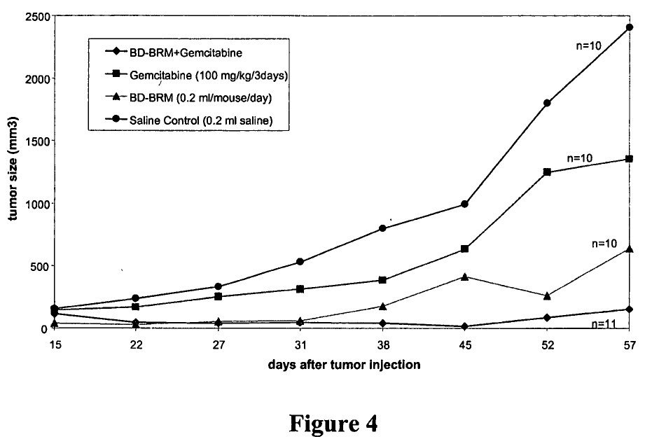

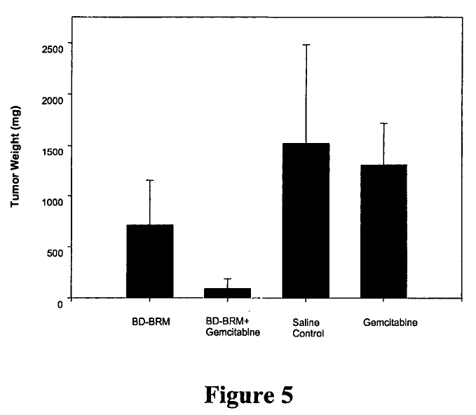

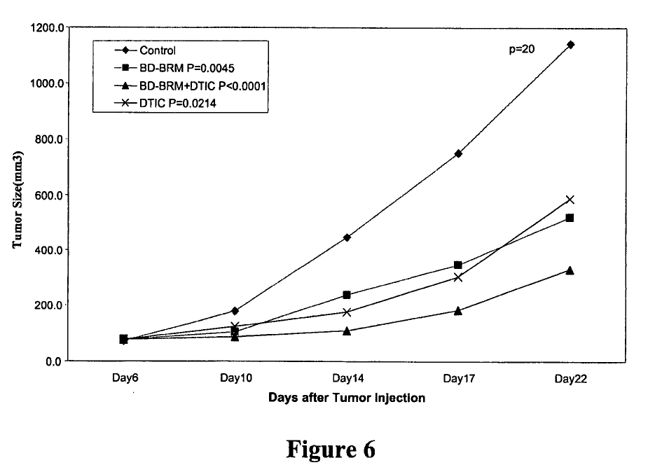

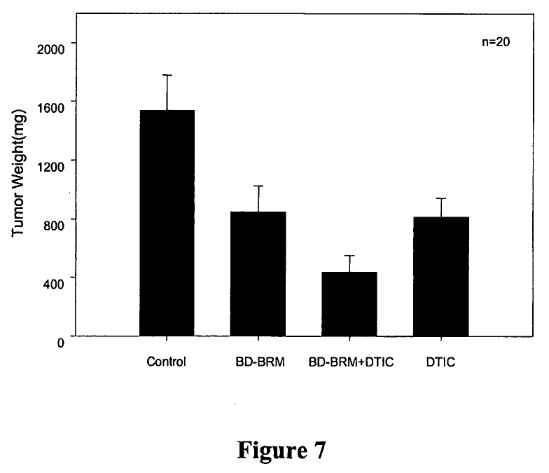

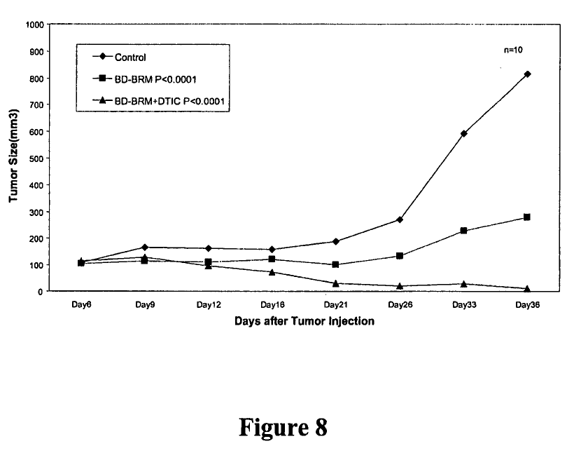

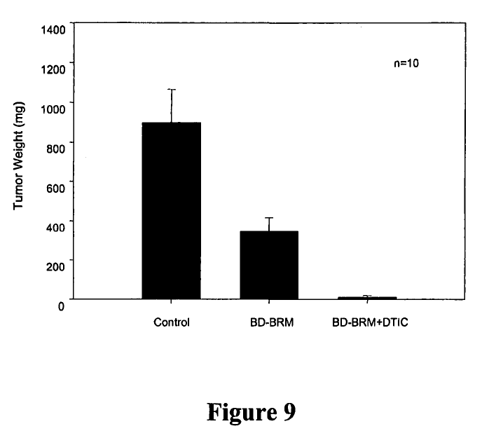

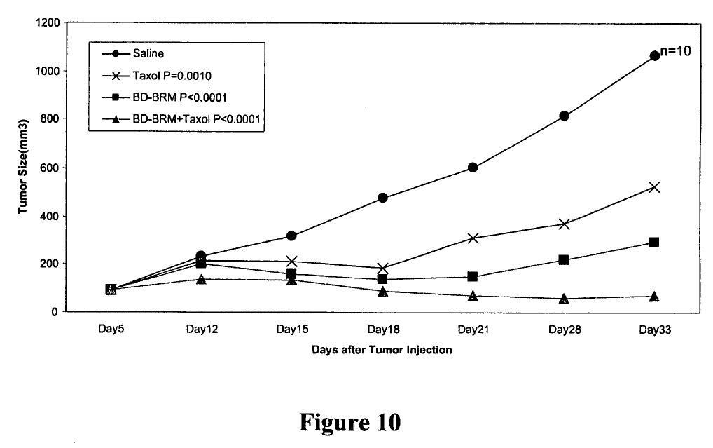

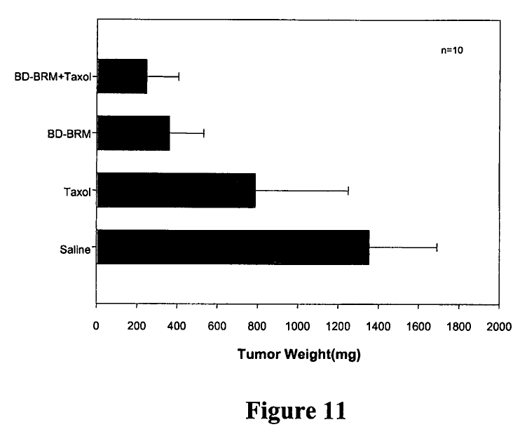

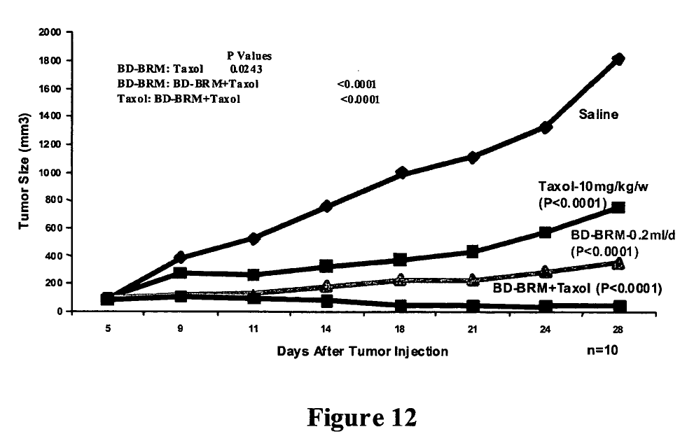

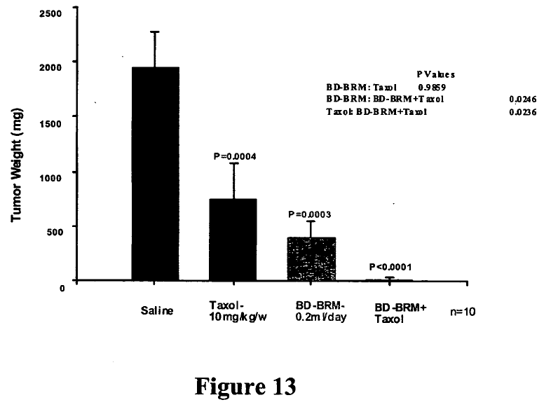

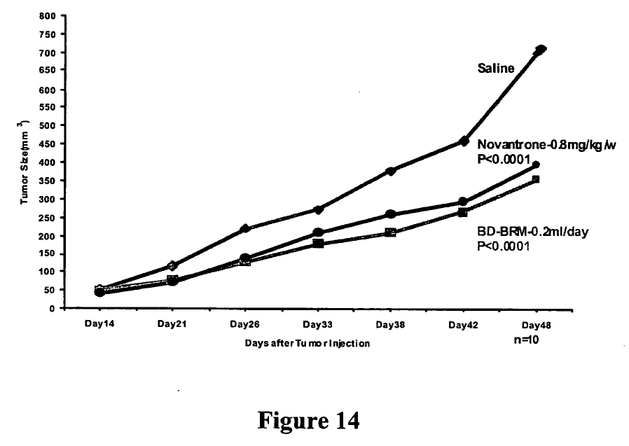

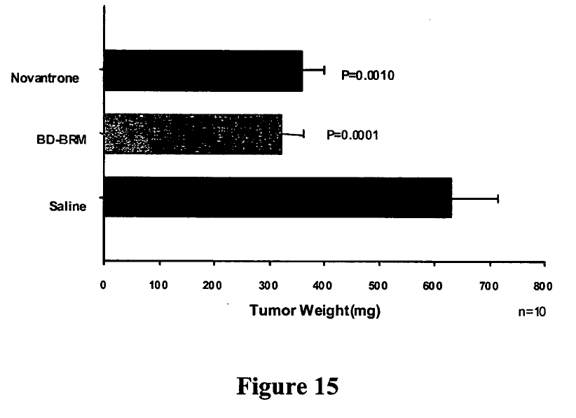

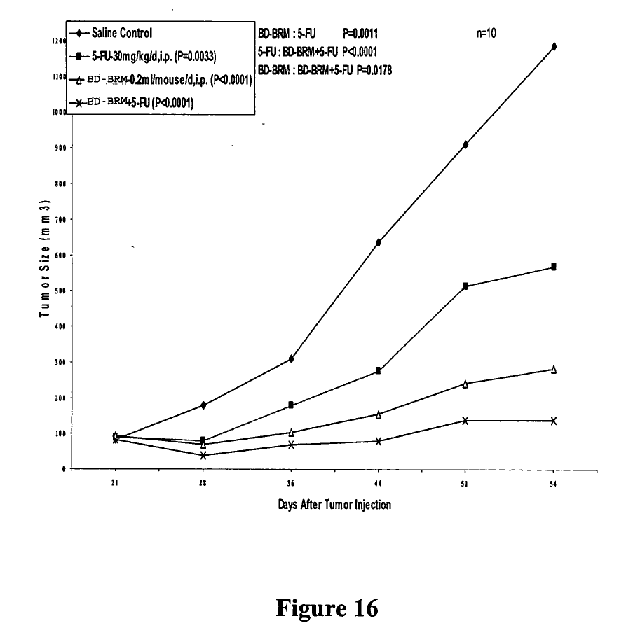

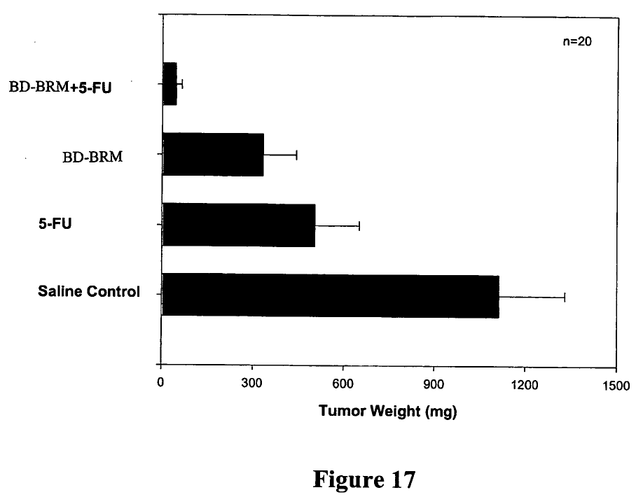

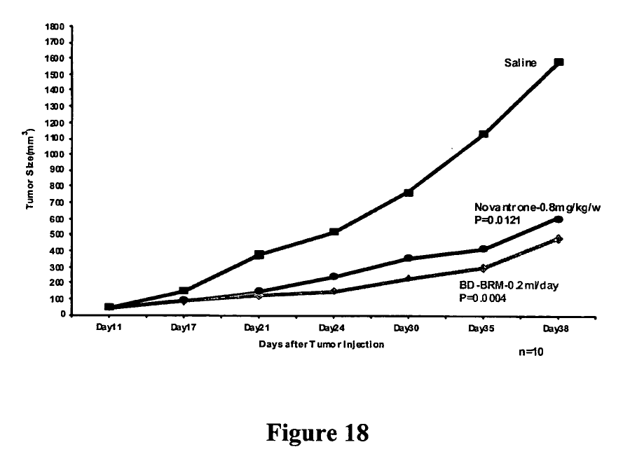

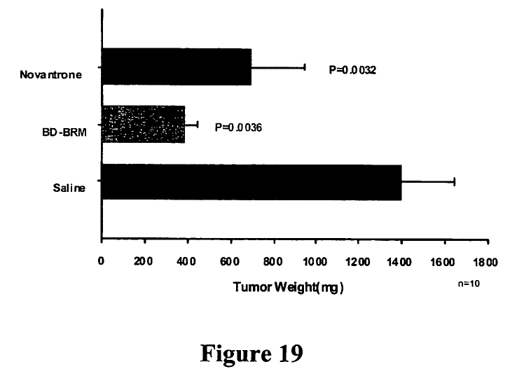

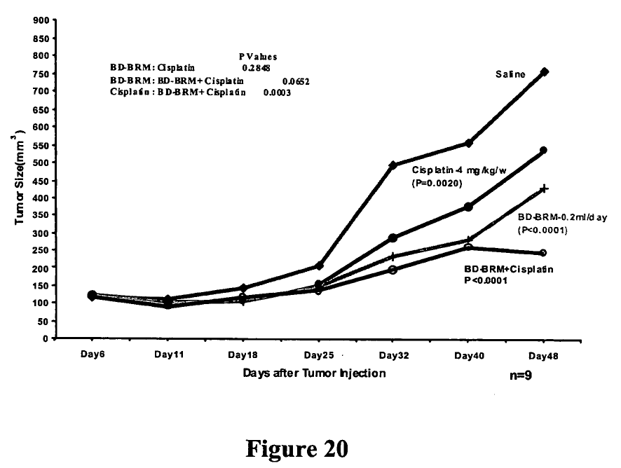

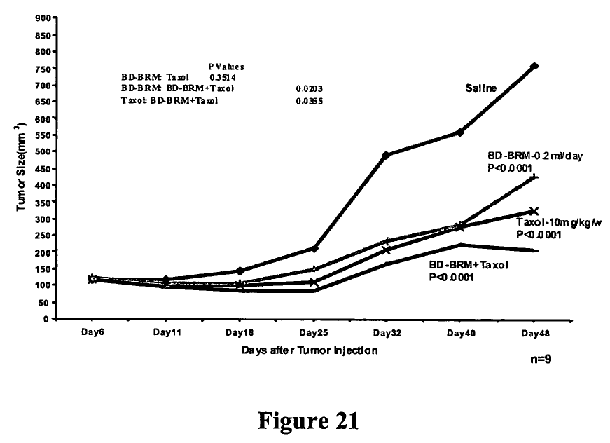

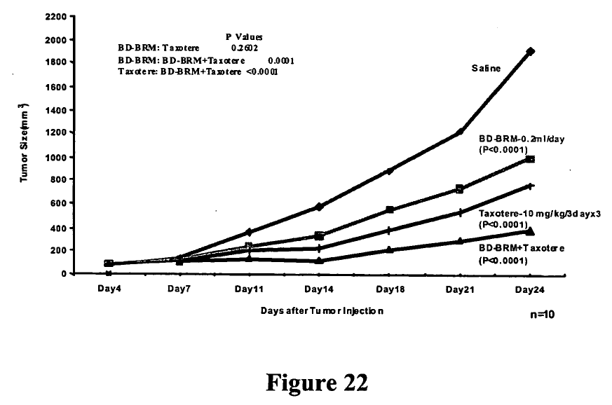

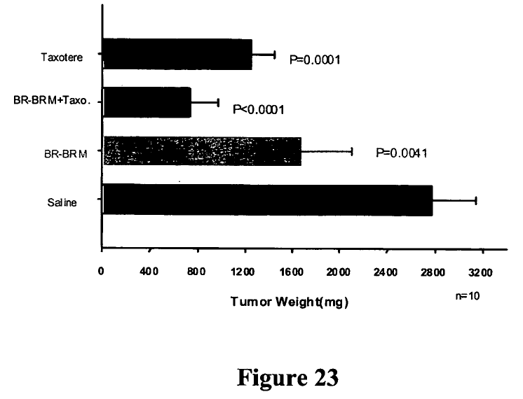

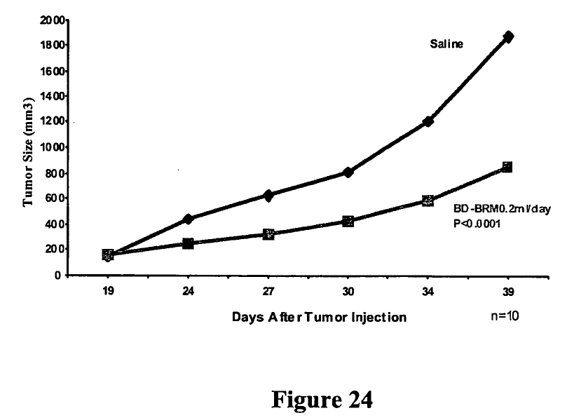

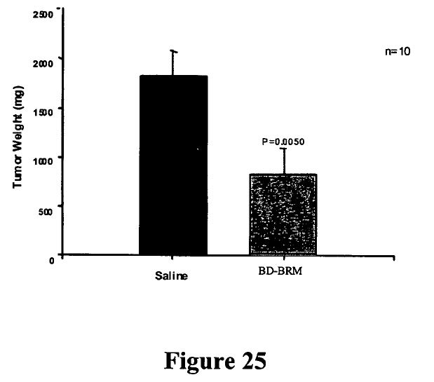

The results of the mouse xenograft experiments outlined in the table above are shown in FIGS. 2-25. BD-BRM treatments always resulted in significant delay of tumor growth compared to saline control. Where a chemotherapeutic drug treatment group was included, the delay in tumor growth achieved with BD-BRM was typically superior to the inhibitory effects observed with the chemotherapeutic drug. As indicated in the above table, total regression of the tumor was also observed in some of the animals, when the animals were treated with a BRM composition alone or with a combination of the BD-BRM composition and a chemotherapeutic drug was used. In the remaining animals treated with a combination, significantly enhanced antitumor effects were observed.

The efficacy of the combinations of the invention can also be determined experimentally using other protocols to study animal models grafted with cancerous cells. The animals subjected to the experiment, can be grafted with a tumor fragment, and the graft may be placed subcutaneously. In the case of the treatment of advanced tumors, tumors are allowed to develop to the desired size, animals having insufficiently developed tumors being eliminated. Animals not bearing tumors may also be subjected to the same treatments as the tumor-bearing animals in order to b able to dissociate the toxic effect from the specific effect on the tumor. Treatment generally begins 3 days to 4 weeks after grafting, depending on the type of tumor, and the animal are observed and animal weight change recorded, and the tumors measured regularly, for example daily, or 2 or 3 times per week until the tumor reaches a defined size (e.g. 2 g in a mouse), or until the animal dies if this occurs before hte tumor reaches 2 g. The animals are autopsied when sacrificed. To study leukemia, cancerous cells can be injected intravenously. Antitumor activity is determined by the increase in the survival time of the treated animals relative to the controls. The efficacy of the treatment with the combination of the invention is assessed in terms of changes in the mean survival time of the animal. Alternative methods of assessing efficacy, and therapeutic synergy, can also be used.

These animal models are recognized in the art to be predictive tests for anticancer effects in humans.

### Example 2

### In Vivo Evaluation of Efficacy of BD-BRM in the Treatment of Human Breast, Ovarian and Prostate Tumors in Mouse Tumor Xenograft Model

Drugs

BD-BRM is an aqueous solution obtained from bovine bile by a standardized process involving solvent extraction and heat hydrolysis. The drug contains 5% (w/v) solid material, comprised of inorganic salts (95-99% of the dry weight) and organic compounds of molecular weights of <3000 daltons (1-5% of the dry weight). BD-BRM is provided as a sterile, injectable formulation. Studies are ongoing to identify all the organic and inorganic components in BD-BRM. Doxorubicin was purchased from Pharmacia and Upjohn (Ontario, Canada), Taxol (Paclitaxel) was from Bristol-Myers Squibb Pharmaceutical (Montreal, Canada), Cisplatin was from Faulding Inc (Quebec, Canada), and Novantrone (Mitoxantrone) was from Wyeth-Ayerst Canada Inc (Montreal, Canada).

Animals and Cells

Mice (CD-1 athymic nude or SCID, 6-8 weeks old, female) were purchased from Charles River Inc. (Montreal, Canada), and maintained in the animal facility of Sunnybrook and Women's College Health Sciences Center (Toronto, Canada). Human tumor cell lines (MDA-MB-231, SK-OV-3, DU145 and PC-3) were from the American Type Culture Collection (ATCC) (Manassas, Va.). Cells were grown in culture media, under conditions recommended in the ATCC technical datasheet. Peritoneal macrophages were isolated from thioglycollate-primed CD-1 mice as described previously⁴, and grown in DMEM culture medium (Wisent, Quebec, Canada), supplemented with 10% FBS and 20% Ladmac supematant⁵. The Ladmac supernatant harvested from Ladmac cell cultures after 5-7 days of incubation provides a source of colony stimulating factor (CSF)-1.

Cytotoxicity Assay

Tumor cells were radio-labeled by the addition of 10 μl/ml of [methyl-³H] Thymidine (5.0 Ci/mmol, Amersham Pharmacia Biotech, Quebec, Canada) into the culture medium. After an overnight incubation the labeled cells were washed four times with PBS. These target tumor cells (5,000 cells per well in a U-bottom 96-well plate) were co-cultured with murine peritoneal macrophages at an effector:target (E/T) cell ratio of 10:1, which was in the plateau range on a curve prepared by varying the E/T ratio from 5:1 to 50:1 (not shown). The co-cultures were grown in DMEM medium supplemented with 10% FBS, 20% Ladmac supernatant and 1 ng/ml rmIFN-γ (eBioscience, CA) in the presence or absence of BD-BRM. in a total volume of 200 μl. After 24 hours incubation at 37° C., the supernatant (150 μl) was collected, and ³H release quantitated using a Beta Spectrometer. The specific cytotoxicity was calculated as follows: specific release (%)=(E−S)/(T−S)×100, in which E is CPM released from target cells in the presence of effector cells; S is CPM released from target cells in the labsence of effector cells and; T is CPM released from target cells after treatment with 10% sodium dodecyl sulfate.

Xenograft Tumor Model

Before reaching approximately 80% confluence in cell culture medium, tumor cells were harvested and resuspended in sterile PBS. Ten million of tumor cells, in 100 μl, were subcutaneously implanted into the right flank of mice (20-28 g body weight). MDA-MB-231 and SK-OV-3 cells were grown in CD-1 athymic nude mice, while DU145 and PC-3 were implanted in SCID mice. The animals were monitored daily. Treatment started when the tumors reached a volume of 50-100 mm³. The animals were randomly separated into groups of at 8-15 animals, so that the mean tumor size distribution was the same in each group. Animals were treated with BD-BRM, saline or standard chemotherapeutic drugs until the endpoint of each experiment. The doses and treatment schedules were as described in the text. All animal experimentation was performed following the National Institutes of Health (USA), Sunnybrook and Women College Health Science Center and Lorus Therapeutics Inc. Animal Care and Use guidelines.

Evaluation of Antitumor Activity

Tumor volume was estimated by caliper measurements, using the formula: length □ width □ height/2⁶. The efficacy of drug treatment was evaluated based on the following calculations: (1) tumor growth inhibition (T/C), calculated as a percentage of the mean tumor volume of drug-treated (T) versus control (C) groups: T/C (%)=(mean tumor volume of drug-treated group/mean tumor volume of control)×100; the optimal value, being the minimal T/C ratio which reflects the maximal tumor growth inhibition achieved⁷; (2) tumor growth delay (T-C), defined as the difference in time for drug-treated (T) and control (C) tumors to reach a given volume (300 mm³); and (3) The final tumor weight (T.W.), determined by the mass of tumor tissue surgically excised from the animal on the last day of the experiment. The percentage of inhibition (%)=(mean tumor weight of controls−mean tumor weight of drug-treated group)/mean tumor weight of controls×100. Statistical analyses, of the differences in tumor weight between treatment groups, were carried out by the Biostatistical Consulting Unit of the Department of Community Health Sciences at the University of Manitoba (Winnipeg, Canada). A p value of ≦0.05 was considered to be statistically significant.

Results

Antitumor efficacy of BD-BRM in human breast, ovarian and prostate tumor xenograft models

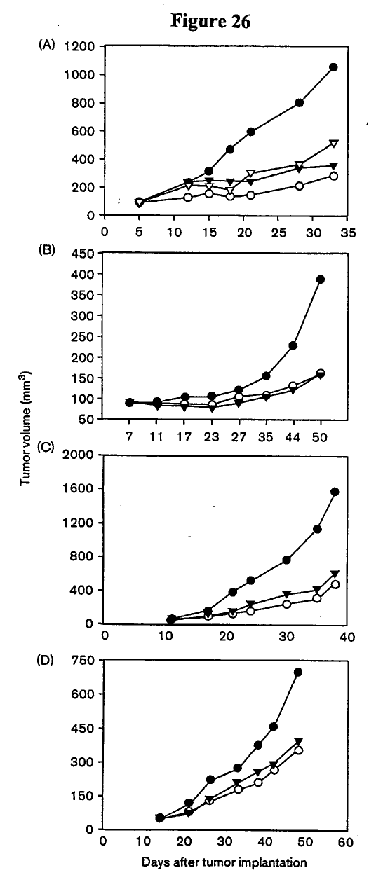

The therapeutic efficacy of BD-BRM, as a single agent, against human tumors (MDA-MB-231, SK-OV-3, DU145 and PC-3) xenografted in mice was tested and compared with standard chemotherapeutic drugs. The results are shown in FIG. 26 and summarized in Table 2. Administration of BD-BRM resulted in a significant delay of MDA-MB-231 breast tumor growth as compared to saline-treated controls (FIG. 26A). This is indicated by a significant optimal T/C value of 24.8% and 20 days of T-C value. The mean tumor weight of BD-BRM -treated animals was decreased by 77% at the endpoint of the experiment as compared to that of saline controls (p=0.0004) (Table 2). In comparison with the tumor growth inhibition by standard chemotherapeutic drug treatments at an optimal dose, 69.4% of tumor weight reduction by Doxorubicin, or 53.2% of tumor weight reduction by Taxol, the efficacy of BD-BRM was higher than these observed in the treatment with either Doxorubicin or Taxol (Table 2).

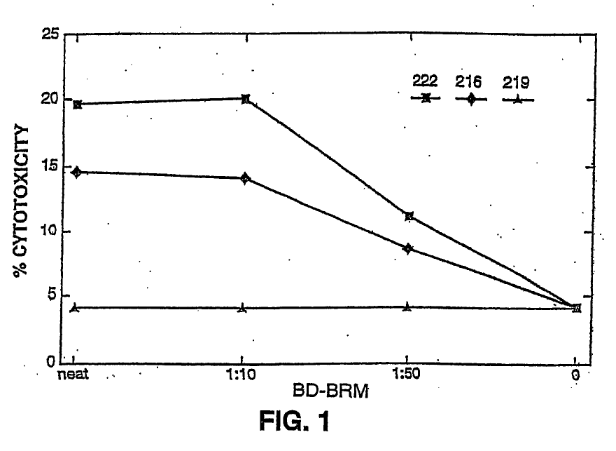

In studies with SK-OV-3, ovarian tumor xenografts, BD-BRM-treatment resulted in a significant delay of tumor growth compared to saline controls (FIG. 1B). This was indicated by a T/C value of 41.2%, with a minor regression from day 17 to 23 (Table 2). The mean tumor weight was decreased significantly by 77.6% in the BD-BRM-treated mice as compared to saline-treated controls p=0.0118), while Cisplatin-treatment demonstrated a similar effect on the mean tumor growth as BD-BRM (FIG. 26B), and the decrease in tumor weight was also statistically significant compared to saline-treated animals (p=0.0443) (Table 2). These results demonstrate that administration of BD-BRM effectively suppresses the growth of SK-OV-3 ovarian tumors in the xenograft mouse model, and that the efficacy of BD-BRM is on par with that of Cisplatin.

The therapeutic effect of BD-BRM on prostate cancer was tested in two prostate tumor models (DU145 and PC-3). In DU145 xenografts (FIG. 26C), BD-BRM treatment retarded tumor growth with an optimal T/C value of 26.1% and 16 days of T-C value, as compared to saline controls (Table 2), indicating a high level of anti-tumor activity against DU145 prostate tumors. The treatment with BD-BRM resulted in 72.6% inhibition in mean tumor weight compared to saline (p=0.0007). The anti-tumor activity of BD-BRM was slightly higher than the conventional drug Novantrone, which had a 50.8% decrease in mean DU145 tumor weight compared to saline control, a 33.4% T/C value, and 8 days of T-C value. To further examine the efficacy of BD-BRM against prostate tumors, BD-BRM was tested against PC-3 prostate tumor xenografts (FIG. 26D). BD-BRM showed a high level of anti-tumor activity against this tumor as indicated by a 50.9% optimal T/C value and 10 days of T-C value (Table 2). The mean tumor weight was decreased by 49.1% in BD-BRM-treated animals as compared to saline-treated controls (p=0.0049). As compared to the treatment with Novantrone, which demonstrated antitumor efficacy with an optimal T/C value of 56.8%, 8 days of T-C and 42.9% of decrease in tumor weight in this PC-3 tumor, again BD-BRM demonstrated superior anti-tumor efficacy compared to Novantrone.

Stimulation Macrophage Cytotoxicity Against Tumor Cells

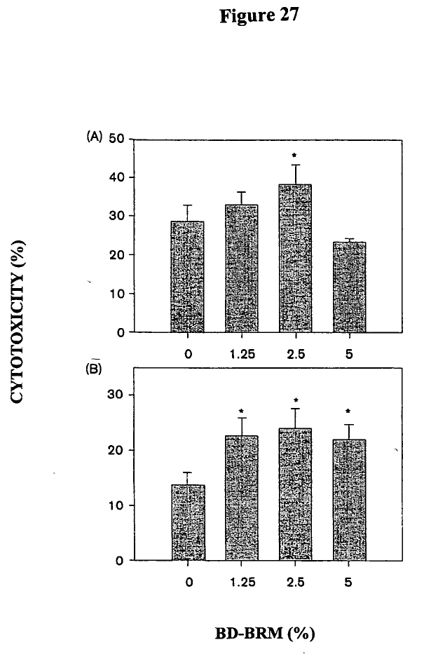

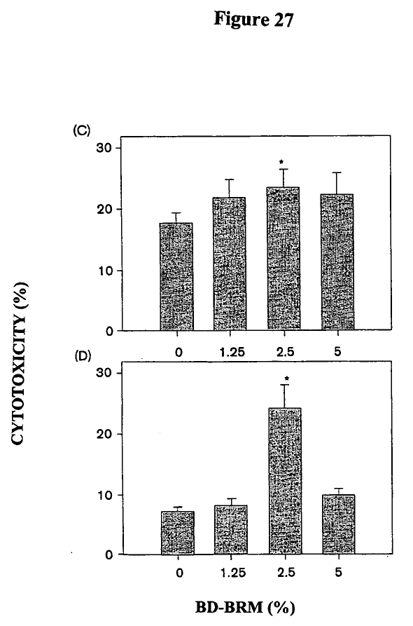

In order to understand the mechanism of BD-BRM anti-tumor action in vivo, the cytotoxicity and immuno-regulatory functions of BD-BRM were examined in vitro. No direct cytotoxic effect or growth retardation was observed with addition of BD-BRM [up to 5% BD-BRM (v/v)] into the culture medium of breast, ovary or prosate tumor cells (data not shown). The potential of BD-BRM to stimulate the cytolytic activity of macrophages against tumor cells was evaluated using a co-culture cytotoxicity assay. As indicated in FIG. 27, the presence of BD-BRM in co-cultures of macrophages with tumor cells stimulated the cytolytic activity of macrophages against all four tumor cell lines. The effect was dose-dependent with a maximum increase in cytotoxicity observed for all cultures at a concentration of 2.5% (v/v) BD-BRM. Macrophage-mediated cytotoxicity in co-cultures with breast tumor MDA-MB-231 cells was 28.5±4.3% in the absence of BD-BRM. This was increased to 38.3±5.1% in the presence of 2.5% (v/v) BD-BRM, an increase of 34.4% (FIG. 27A). Similarly, the cytotoxicity in co-cultures with ovarian tumor SK-OV-3 cells, in the presence of 2.5% (v/v) BD-BRM, was increased by 72.5% compared to that of cultures in the absence of BD-BRM(from 13.8±2.2% in the absence of BD-BRM to 23.8±3.7% in the presence of BD-BRM) (FIG. 27B). In co-cultures of macrophages with prostate tumor cells, an increase of 32.8% in macrophage-mediated cytotoxicity (from 17.7±1.7% in the absence of BD-BRM to 23.5±3.0% in the presence of 2.5% BD-BRM) was observed against DU145 prostate tumor cells (FIG. 27C), while macrophage-mediated cytotoxicity increased more than 300%, from 7.3±0.7% (absence of BD-BRM) to 24.2±3.9% (2.5% BD-BRM), against PC-3 prostate tumor cells (FIG. 27D).

TABLE 2
Summary of BD-BRM efficacy agains xenografted human breast,
ovarian and prostatic tumors

| | | Optimum T/C (%) | T − C (days) or | Mean tumor weight | Inhibition | p value |
|---|---|---|---|---|---|---|
| Tumor | Drug (n = 10) | [day] | MR [period] | (mg) | (%) | (versus saline) |
| MDA-MB-231 | saline | | | 1649 | | |
| | BD-BRM | 24.8 [21] | 20 | 378.8 | 77 | 0.0004 |
| | doxorubicin | 34.4 [33] | 10 | 505.3 | 69.4 | 0.0008 |
| | taxol | 38.6 [18] | 7 | 771 | 53.2 | 0.0107 |
| SK-OV-3 | saline | | | 500 | | |
| | BD-BRM | 41.2 [49] | MR [17-23] | 112 | 77.6 | 0.0118 |
| | cisplatin | 41.7 [49] | MR [17-23] | 195 | 61 | 0.0443 |
| DU145 | saline | | | 1401 | | |
| | BD-BRM | 26.1 [35] | 16 | 381 | 72.6 | 0.0007 |
| | novantrone | 33.4 [30] | 8 | 690 | 50.8 | 0.0295 |
| PC-3 | saline | | | 629 | | |
| | BD-BRM | 50.9 [48] | 10 | 320 | 49.1 | 0.0049 |
| | novantrone | 56.8 [48] | 8 | 359 | 42.9 | 0.0141 |

Body weight was not observed in any animal treated with saline or BD-BRM alone. The tumor growth delay (T − C) was calculated at mean tumor size of 300 mm³.
MR = minor regression.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

### REFERENCES

4. Mishell B B, Shiigi S M. Selected methods in cellular immunology. San Francisco: Freeman, 1980.
5. Du C, Sriram S. Induction of interleukin-12/p40 by superantigens in macrophages is mediated by activation of nuclear factor-□B. Cell. Immunol. 2000; 199: 50-57.
6. Tomayko M M, Reynolds C P. Determination of subcutaneous tumor size in athymic (nude) mice. Cancer Chemother. Pharmacol. 1989; 24: 148-154.
7. Hendriks H R, Langdon S, Berger D P, et al. Comparative antitumor activity of vinblastine-isoleucinate and related vinca alkaloids in human tumor xenografts. Eur. J. Cancer 1992; 28A: 767-773.
8. MacDonald H R, Lees R K. Frequency and specificity of precursor of interleukin 2-producing cells in nude mice. J. Immunol. 1984; 132: 605-610.
9. MacDonald H R, Blanc C, Lees R K, Sordat B. Abnormal distribution of T cell subsets in athymic mice. J. Immunol. 1986; 136: 4337-4339.
10. Bosma G C, Custer R P, Bosma M J. Severe combined immunodeficiency mutation in the mouse. Nature 1983; 302: 527-530.
11. Silobrcic V, Zietman A L, Ramsay J R, Suit H D, Sedlacek R S. Residual immunity of athymic NCr/Sed nude mice and the xenotransplantation of human tumors. Int. J. Cancer 1990; 45: 325-333.
12. Wu T Y, Fleischmann W R Jr. Murine B16 melanoma vaccination-induced tumor immunity: identification of specific immune cells and functions involved. J. Interferon Cylokine Res. 2001; 21: 1117-1127.
13. Adams D O, Hall T, Steplewski Z, Koprowski H. Tumors undergoing rejection induced by monoclonal antibodies of the IgG2a isotype contain increased numbers of macrophages activated for a distinctive form of antibody-dependent cytolysis. Proc. Natl. Acad. Sci. USA 1984; 81: 3506-3510.
14. Shetye J, Frodin J E, Christensson B, et al. Immunohistochemical monitoring of metastatic colorectal carcinoma in patients treated with monoclonal antibodies (Mad 17-1A). Cancer Immunol. Immunother. 1988; 27: 154-162.
15. Masucci G, Wersall P, Ragnhammar P, Mellstedt H. Gradulocyte-monocyte-colony-stimulating factor augments the cytotoxic capacity of lymphocytes and monocytes in antibody dependent cellular cytotoxicity. Cancer Immunol. Immunother. 1989; 29: 288-292.
16. Dranoff G, Jaffee E, Lazenby A, et al. Vaccination with irradiated tumor cells engineered to secrete murine granulocyte-macrophage colony-stimulating factor stimulates potent, specific, and long-lasting anti-tumor immunity. Proc. Natl. Acad. Sci. USA 1993; 90: 3539-3543.
17. Small E J, Reese D M, Um B, Whisenant S, Dixon S C, Figg W D. Therapy of advanced prostste cancer with granulocyte-macrophage colony-stimulating factor. Clin. Cancer Res. 1999; 5: 1738-1744.
18. James N D, Atherton P J, Jones J, Howie A J, Tchekmedyian S, Curnow R T. A phase II study of the bispecific antibody MDX-H210 (anti-HER2×CD64) with GM-CSF in HER2⁺ advanced prostate cancer. Br. J. Cancer 2001; 85: 152-156.
19. Reed E, Janik J, Bookman M A, et al. High-dose carboplantin and recombinant granulocyte macrophage colony-stimulating factor in advanced-stage recurrent ovarian cancer. J. Clin. Oncol. 1993; 11: 2118-2126.
20. McClay E F, Bradly P D, Kirmani S, et al. A phase II trial of intraperitoneal high-dose carboplatin and etoposide with granulocyte macropahge-colony stimulating factor support in patients with ovarian carcinoma. Am. J. Clin. Oncol. 1995; 18: 23-26.
21. Morello E, Dernell W S, Kuntz C A, et al. Evaluation of Cisplatin in combination with a Biological Response Modifier in a murine mammary carcinoma model. Cancer Invest. 2002; 20: 480-489.

1. (canceled)

2. Use of a composition in the manufacture of a medicament for the treatment of breast or prostate cancer in a mammal, said composition comprising small molecular weight components of less than 3000 daltons, and having the following properties:
(a) is extracted from bile of animals;
(b) is capable of stimulating monocytes and/or macrophages in vitro and/or in vivo;
(c) is capable of modulating tumor necrosis factor production and/or release;
(d) contains no measurable level of IL-1α, IL-1β, TNF, IL-6, IL-8, IL-4, GM-CSF or IFN-gamma;
(e) is not cytotoxic to human peripheral blood mononuclear cells; and
(f) is not an endotoxin.

3. A pharmaceutical kit for the treatment of breast or prostate cancer in a mammal, said kit comprising:
(1) a dosage unit of a composition and a pharmaceutically acceptable carrier wherein the composition comprises small molecular weight components of less than 3000 daltons, and has the following properties:
(a) is extracted from bile of animals;
(b) is capable of stimulating monocytes and/or macrophages in vitro and/or in vivo;
(c) is capable of modulating tumor necrosis factor production and/or release;
(d) contains no measurable level of IL-1α, IL-1β, TNF, IL-6, IL-8, IL-4, GM-CSF Or IFN-gamma;
(e) is not cytotoxic to human peripheral blood mononuclear cells;
(f) is not an endotoxin; and
(2) a dosage unit of one or more chemotherapeutic drug(s) and a pharmaceutically acceptable carrier,
said (1) and (2) being provided in amounts that that are effective in combination for killing tumour or metastatic cells.

4. The kit according to claim 3, wherein said one or more chemotherapeutic drug(s) is gemcitabine, 5-fluorouracil, dacarbazine, taxol, taxotere, cisplatin or mitoxantrone.

5. A pharmaceutical composition for the treatment of breast or prostate cancer in a mammal, said pharmaceutical composition comprising:
(1) a composition comprising small molecular weight components of less than 3000 daltons, and having the following properties:
(a) is extracted from bile of animals;
(b) is capable of stimulating monocytes and/or macrophages In vitro and/or in vivo;
(c) is capable of modulating tumor necrosis factor production and/or release;
(d) contains no measurable level of IL-1α, IL-1β, TNF, IL6, IL-8, IL-4, GM-CSF or IFN-gamma;
(e) is not cytotoxic to human peripheral blood mononuclear cells;
(f) is not an endotoxin;
(2) one or more chemotherapeutic drug(s); and
(3) a pharmaceutically acceptable carrier;
wherein said pharmaceutical composition has therapeutic synergy or improves the therapeutic index in the treatment of cancer over the composition or the chemotherapeutic drug(s) alone.

6. The pharmaceutical composition according to claim 5, wherein at least one of said one or more chemotherapeutic drug(s) is gemcitabine, 5-fluorouracil, dacarbazine, taxol, taxotere, cisplatin or mitoxantrone.

7. The pharmaceutical composition according to claim 5, formulated into a sterile solution, a lyophilate, a pill, a tablet, a cream, a capsule, a suppository, a gelatin capsule, a soft gelatin capsule, a gel, a membrane or a tubelet.

8. (canceled)

9. (canceled)

10. (canceled)

11. (canceled)

12. (canceled)

13. The pharmaceutical composition according to claim 5, wherein said cancer is breast cancer and said anticancer agent is taxol.

14. The pharmaceutical composition according to claim 5, wherein said pharmaceutical composition is suitable for administration via oral, topical, rectal, parenteral, local, inhalant or intacerebral delivery.

15. The pharmaceutical composition according to claim 14, wherein said parenteral delivery is achieved via intramuscular injection.

16. Use of a combination for the manufacture of a medicament for the treatment of breast or prostate cancer in a mammal, said combination comprising:
(1) a composition comprising small molecular weight components of less than 3000 daltons, and having the following properties:
(a) is extracted from bile of animals;
(b) is capable of stimulating monocytes and/or macrophages in vitro and/or in vivo;
(c) is capable of modulating tumor necrosis factor production and/or release;
(d) contains no measurable level of IL-1α, IL-1β, TNF, IL-6, IL-8, IL-4, GM-CSF or IFN-gamma;
(e) is not cytotoxic to human peripheral blood mononuclear cells;
(f) is not an endotoxin; and
(2) one or more anticancer agent(s),
wherein said combination has therapeutic synergy or improves the therapeutic index in the treatment of cancer over the composition or the anticancer agent(s) alone.

17. A method for treating breast or prostate cancer in a mammal, comprising administering to said mammal a therapeutically effective amount of a composition comprising small molecular weight components of less than 3000 daltons, and having the following properties:
(a) is extracted from bile of animals;
(b) is capable of stimulating monocytes and/or macrophages in vitro and/or in viva;
(c) is capable of modulating tumor necrosis factor production and/or release;
(d) contains no measurable level of IL-1α, IL-1β, TFN, IL-6, IL-8, IL-4, GM-CSF or IFN-gamma;
(e) is not cytotoxic to human peripheral blood mononuclear cells;
(f) is not an endotoxin.

18. A method for treating breast or prostate cancer in a mammal, comprising administering to said mammal a therapeutically effective amount of a composition and one or more anticancer agent(s), wherein said composition comprises small molecular weight components of less than 3000 daltons, and has the following properties:
(a) is extracted from bile of animals;
(b) is capable of stimulating monocytes and/or macrophages in vitro and/or in vivo;
(c) is capable of modulating tumor necrosis factor production and/or release;
(d) contains no measurable level of IL-1α, IL-1β, TNF, IL-6, IL-8, IL-4, GM-CSF or IFN-gamma;
(e) is not cytotoxic to human peripheral blood mononuclear cells;
is not an endotoxin.

19. The method according to claim 18, wherein said anticancer agent(s) is selected from the group consisting of a chemotherapeutic drug, radiation, a gene therapy and an antisense oligonucleotide.

20. The method according to claim 19, wherein said anticancer agent(s) is a chemotherapeutic drug, an interleukin or an interferon.

21. The method according to claim 18, wherein at least one of said one or more anticancer agent(s) is a chemotherapeutic drug.

22. The method according to claim 21, wherein the chemotherapeutic drug is gemcitabine, 5-fluorouracil, dacarbazne, taxol, taxotere, cisplatin or mitoxantrone.

23. The method according to claim 17 or 18, wherein said composition or combination is formulated into a sterile solution, a lyophilate, a pill, a tablet, a cream, a capsule, a suppository, a gelatin capsule, a soft gelatin capsule, a gel, a membrane or a tubelet.

24. The method according to claim 17 or 18, wherein said administering is achieved by means of oral, topical, rectal, parenteral, local, inhalant, or intracerebral delivery.

25. The method of claim 24, wherein said parenteral delivery is achieved via intramuscular injection.

26. The method of claim 17 or 18, wherein peripheral blood monocytes and/or tumor associated macrophages are stimulated to express cytocidal activity in a manner that is insensitive to the inhibitory effects of prostaglandins.

27. The method of claim 17 or 18, wherein suitable modulation of the immune system is elicited in said mammal by activating macrophages and/or monocytes to produce and/or release cytokines or promote activity to seek and remove or destroy cancerous cells.

28. The method of claim 17 or 18, wherein the release of TNF, Il-1β and GM-CSF is stimulated in said mammal.

29. Use of a composition in the manufacture of a pharmaceutical kit for the treatment of breast or prostate cancer in a mammal, said kit comprising:
(1) a dosage unit of a composition and a pharmaceutically acceptable carrier wherein the composition comprises small molecular weight components of less than 3000 daltons, and has the following properties:
(a) is extracted from bile of animals;
(b) is capable of stimulating monocytes and/or macrophages in vitro and/or in vivo;
(c) is capable of modulating tumor necrosis,factor production and/or release;
(d) contains no measurable level of IL-1α, IL-1β, TNF, IL-6, IL-8, IL-4, GM-CSF or IFN-gamma;
(e) is not cytotoxic to human peripheral blood mononuclear cells;
(f) is not an endotoxin; and
(2) a dosage unit of one or more chemotherapeutic drug(s) and a pharmaceutically acceptable carrier,
said (1) and (2) being provided in amounts that have therapeutic synergy or that improve the therapeutic index in the treatment of cancer over the composition or the chemotherapeutic drug(s) alone.

30. A pharmaceutical kit for the treatment of breast or prostate cancer in a mammal, said kit comprising:
(1) a dosage unit of a composition and a pharmaceutically acceptable carrier wherein the composition comprises small molecular weight components of less than 3000 daltons, and has the following properties:
(a) is extracted from bile of animals;
(b) is capable of stimulating monocytes and/or macrophages in vitro and/or in vivo;
(c) is capable of modulating tumor necrosis factor production and/or release;
(d) contains no measurable level of IL-1α, IL-1β, TNF, IL-6, IL-8, IL-4, GM-CSF or IFN-gamma;
(e) is not cytotoxic to human peripheral blood mononuclear cells;
(f) is not an endotoxin; and
(2) a dosage unit of one or more chemotherapeutic drug(s) and a pharmaceutically acceptable carrier,
said (1) and (2) being provided in amounts have therapeutic synergy or that improve the therapeutic index in the treatment of cancer over the composition or the chemotherapeutic drug(s) alone.

31. The method according to claim 18, wherein said composition and said one or more anticancer agent(s) are administered separately, concurrently or simultaneously.

32. The method according to claim 18, wherein said cancer is breast cancer and said anticancer agent is taxol.

33. The method according to claim 32, wherein said composition and said anticancer agent are administered concurrently.
